(19) United States
(12) Patent Application Publication
(10) Pub. No.: US 2003/0229335 A1
(43) Pub. Date: Dec. 11, 2003

(54) Apparatus and method for delivery of constrained beneficial bacteria to the vaginal tract

(76) Inventors: Richard Grady Payne, Alpharetta, GA (US); Jaeho Kim, Roswell, GA (US); Robert John Lyng, Alpharetta, GA (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/137,090

(22) Filed: Apr. 30, 2002

(51) Int. Cl. A61K 45/00; A61K 9/22

(52) U.S. Cl. 604/890.1; 424/93.4

(57) ABSTRACT

The present invention relates to a treatment for vaginal infections that does not promote colonization of artificially introduced bacteria such as Lactobacillus. Beneficial bacteria are contained within an outer cover of a vaginal insert that does not allow the bacteria to contact the vaginal tissue. The outer cover allows passage of metabolic byproducts produced by the beneficial bacteria into the vaginal tract creating an environment hostile to pathogenic microorganisms. The vaginal insert is placed in the vaginal tract by the user either by hand or by an instrument such as a tampon applicator. When the treatment time elapses the vaginal insert is removed.

### FIELD OF THE INVENTION

This invention relates to a vaginal insert for delivery of beneficial bacteria and beneficial bacterial metabolic byproducts to the vaginal tract. Beneficial bacteria are contained in a vaginal insert and only the bacterial byproducts are capable of exiting the insert, thereby allowing the vaginal tract to receive the benefits of the bacteria without promoting colonization of the artificially introduced bacteria in the vaginal tract.

### BACKGROUND OF THE INVENTION

The human urogenital system contains naturally occurring non-pathogenic bacteria. A normal vagina houses non-pathogenic bacteria and small amounts of yeast. Often, however, the normal balance of microbial flora in the normal, healthy vaginal tract is disrupted by organisms introduced from the gastrointestinal system, use of antibiotics, various health issues, or even increased sexual activity.

Bacterial vaginosis and yeast infections are two possible results from an upset in the balance in the vaginal tract. These conditions are characterized by a smelly secretion and, sometimes, slight itching sensations. Bacteria vaginosis and yeast infections occur frequently during pregnancy and while these conditions are troublesome to nonpregnant women, they are thought to increase risk of premature, low-birth weight infants.

One method of treating pathogenic microorganism infections of the vaginal tract is to introduce and/or promote colonization of the naturally occurring microorganisms. Use of Lactobacilli such as Lactobacillus acidophilus to treat vaginal infections is well known. Lactobacilli are gram positive bacteria that are part of the normal, healthy microbial flora of the vagina as well as the mouth and digestive system. Lactobacilli help fight vaginal infections by producing metabolic byproducts such as hydrogen peroxide (H₂O₂) and creating an acidic environment.

Typical Lactobacillus treatments artificially introduce Lactobacilli or other beneficial bacteria into the vaginal tract with the intention that these introduced bacteria will adhere to the vaginal tissue and result in new beneficial bacterial colonies. Introduction of this method of bacterial treatment is typically done by oral supplements, a vaginal cream, or by a vaginal suppository. The lactobacilli are inactive in the treatment vehicle and activated upon entering the body.

The treatment success is dependent upon colonization of the lactobacilli, which can be difficult to obtain due to the existing hostile environment. Prolonged use of the treatment may be necessary and may result in an apprehension of the potential user given the nature of the treatment product. Also, many people today are concerned about using too many medications. In some cases it was using medication such as antibiotics which may have lead to the vaginal infection. Therefore there is a need for vaginal treatments that do not introduce artificial colonization of Lactobacilli for those who prefer to encourage the natural course of healing of vaginal infections.

### SUMMARY OF THE INVENTION

The present invention relates to a treatment for vaginal pathogenic bacteria and/or yeast infections that does not promote artificial colonization of beneficial bacteria such as Lactobacilli on the vaginal tissue. The beneficial bacteria are contained within an outer cover of a vaginal insert that does not allow the bacteria to contact the vaginal tissue. The outer cover allows passage of metabolic byproducts produced by the beneficial bacteria into the vaginal tract creating an environment hostile to pathogenic microorganisms.

The bacteria source is inactivated by lyophilization (freeze-drying) or other known methods. The freeze-dried bacteria can be in a powder form within the outer cover or encapsulated in hydrogel microspheres. The outer cover material is permeable to metabolic byproducts but not to the free bacteria or encapsulated bacteria. If the bacteria source is in a powder form the outer cover will be a microporous membrane made of various materials such as cellulose. If the bacteria are encapsulated the outer cover can have a larger pore size which allows for use of additional materials including natural cloths.

The inactive bacteria source can be mixed with a dried food source to promote growth/metabolic processes when activated just prior to use or during use. The food source may be in solution and stored in a plastic blister package either within the outer cover or packaged in relation to the vaginal insert such that when the blister is squeezed just prior to use the bacteria source is rehydrated/reactivated and metabolism begins.

The vaginal insert is placed in the vaginal tract by the user either by hand or by an instrument such as a tampon applicator. When the treatment time elapses the vaginal insert is removed. A string can be attached to the outer cover to aid in removal.

### BRIEF DESCRIPTION OF THE DRAWINGS

FIG. 1 is a cross sectional view of a vaginal insert according to one embodiment of this invention.

FIG. 2 is a cross sectional view of a vaginal insert according to one embodiment of this invention.

FIG. 3 is a plan view of blister packaged vaginal insert according to one embodiment of this invention.

### DEFINITIONS

Within the context of this specification, each term or phrase below will include the following meaning or meanings.

“Beneficial bacteria” refers to bacteria that occurs naturally in the human body. However, bacteria beneficial to one body system such as the gastrointestinal system may not be beneficial to another body system such as the urogenital system. Beneficial bacteria useful in this invention are bacteria that are naturally occurring in the vagina/vaginal tract. “Beneficial bacteria” also includes non-naturally occurring bacteria that will produce an environment in the vagina or vaginal tract that is conducive to eliminating pathogenic bacteria and/or promoting naturally occurring bacteria.

“Blister pack” or “blister” refer to a packaging device wherein the item to be packaged is surrounded, at least in part, by plastic. The plastic enclosure resembles a blister. Blister packs are often used to package consumer medicines such as decongestants or pain relievers.

“Lactobacillus” or “Lactobacilli” refers to species of bacteria classified under the genus Lactobacillus. There are numerous species and strains of Lactobacilli. Lactobacilli generally metabolize sugars such as lactose and produce byproducts including lactic acid and hydrogen peroxide. This invention can use any species/strain of Lactobacillus depending on the needs of the user.

“Microsphere” refers to a colloidal conglomerate formed as a dispersed phase of a colloidal system. Microspheres can be formed as a hydrogel by dripping a bacteria solution suitable to form a dispersed phase into a dispersing medium solution (the continuous phase).

“Pathogenic bacteria” or “pathogenic microorganisms” refers to microbes that are not a natural part of a healthy body system. Pathogenic organisms cause infection, illness, and other serious conditions that generally require treatment. Organisms can be naturally occurring in a first body system such as the gastrointestinal system and pathogenic in a second body system such as the urogenital system.

“Nonwoven” refers to materials formed without the aid of a textile weaving or knitting process.

These terms may be defined with additional language in the remaining portions of the specification.

### DETAILED DESCRIPTION OF THE PRESENTLY PREFERRED EMBODIMENTS

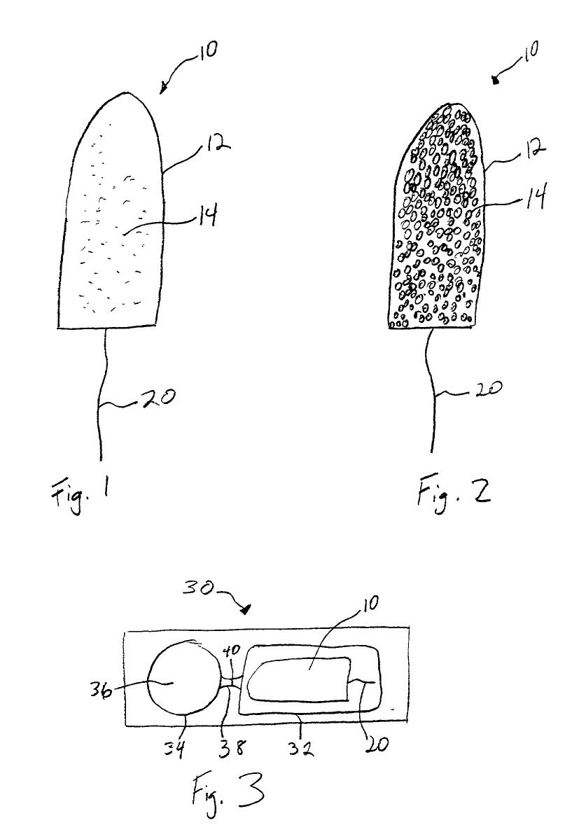

FIG. 1 shows a vaginal insert 10 useful in treating infections of the vaginal tract. The vaginal insert 10 includes a bacteria source 14 surrounded by an outer cover 12. The outer cover 12 is impermeable to the bacteria source 14, yet is permeable to beneficial metabolic byproducts produced by the bacteria source 14. The outer cover 12 is typically a porous membrane having a pore size relative in size to the bacteria source 14 so as to not allow bacteria from the bacteria source 14 to permeate the outer cover 12.

FIG. 1 shows the bacteria source 14 in a free-form bacterial powder. When the bacteria source 14 is in a powder form, the outer cover 12 is a microporous membrane. Microporous membranes useful in this invention will have a pore size desirably smaller than the bacteria cell diameter, thus being impermeable to the bacteria source 14. For example most Lactobacillus cells are about 1-3 micrometers in diameter; therefore using a membrane with a pore size of less than about 1 micrometer would be sufficient to be impermeable to the bacteria source 14. In one embodiment of this invention it is desirably that the outer cover 12 be impermeable to substantially all of the bacteria source 14, although a small number of individual bacterial cells may permeate through the outer cover 12. One test for permeability and pore size of the microporous membrane useful in this invention is ASTM Test Method E1294-89 (1999) “Standard Test Method for Pore Size Characteristics of Membrane Filters Using Automated Liquid Porosimeter,” herein incorporated by reference.

In one embodiment of this invention the microporous membrane is made from cellulose. Various types of cellulose include wood pulp fibers, cotton fibers, and other plant fibers which can be formed into porous nonwoven webs. Other nonwoven webs can also be used for the microporous membrane outer cover 12 including spunbond webs, meltblown webs, carded fiber webs, air laid webs, and the like, made from thermoplastic materials such as polyolefin (e.g. polyethylene and polypropylene homopolymers and copolymers), polyesters, polyamines, polytetrafluoroethylene, and the like. Microporous films made from these thermoplastic materials can also be employed. Dialysis membranes made from cellulose, cellulose acetate, benzolyated cellulose, polyacrylonitrile, and other materials, can also form the microporous membrane outer cover 12. Microporous membrane materials and construction are generally known in the art.

In one embodiment of this invention, the outer cover 12 is made from a sturdy material that will retain its structural integrity during application, use, and removal from the vaginal tract. Cellulose outer covers generally provide the desired characteristics. In another embodiment of this invention the vaginal insert 10 can use a backbone member (not shown) that adds additional support for the vaginal insert. FIG. 1 shows a thimble shaped vaginal insert 10, however there is no limitation as to the actual shape of vaginal inset 10. Circular or oval vaginal inserts, for instance, can also be used. As will be appreciated by one skilled in the art following the teachings herein, the size and shape of the vaginal insert 10 can vary depending on need and use.

A string 20 is attached to the outer cover 12 and used for removal of vaginal insert 10 after treatment is finished. The use and construction of the string 20 are well known in the art and the string 20 operates in the same manner as with other feminine products such as tampons.

In one embodiment of this invention, the bacteria source 14 is in an inactive state in the vaginal insert 10 and can be activated just prior to use. The bacteria source 14 can be inactivated by methods known in the art such as lyophilization (freeze-drying) or sporulation. Various freeze-drying methods known in the art can be used to inactivate the bacteria source 14. One such freeze-drying method includes flash freezing the bacteria in liquid nitrogen followed by vacuum sublimation using a freeze-dryer. Sporulation is generally known in the art and is generally available only for certain bacteria that will sporulate.

FIG. 2 shows another embodiment of the vaginal insert 10 according to this invention. In FIG. 2, the bacteria source 14 is encapsulated in microspheres of bacteria. “Encapsulated” refers to bacteria cell microspheres which have a larger size than individual bacterial cells in powder form. In one embodiment of this invention, the bacteria source 14 is encapsulated in a gel or polymer microparticle. Microparticles of this invention can be formed, without limitation, using alginate, gelation, other hydrogel forming materials, and by formation of polymer shells using polymers such as poly(hydroxyethylmethacrylate). Encapsulated bacteria maintain the microsphere form both in the inactive state and after reactivation for use.

In one embodiment encapsulation is obtained by mixing free form bacteria or bacterial broth into a 1.5 percent by weight alginate solution, such as alginic acid, sodium salt available from Fluka BioChemika, Switzerland, designated as product number 71238. The alginate solution is then dripped into a 1.5 percent by weight calcium carbonate solution. When added to the 1.5 percent by weight calcium carbonate solution, the alginate solution forms microspheres which are then filtered to remove the alginate microspheres from the calcium carbonate solution. The bacterial alginate microspheres are then inactivated by freeze-drying or other inactivating process.

In one embodiment of this invention the bacteria source 14 is encapsulated and the outer cover 12 is a cloth-like cover. Encapsulating the bacteria source 14 creates microspheres having a larger size than the individual bacterial cells, and because the encapsulated microspheres generally maintain structural integrity throughout use, a wider range of cover materials can be used for the outer cover 12. Cloths with larger pores, made of natural fibers, nonwoven fibers, and combinations thereof, are useful with encapsulated bacteria. Microporous membranes can also be used with an encapsulated bacteria source.

The bacteria source 14 preferably contains beneficial bacteria. “Beneficial bacteria” refers to the naturally occurring bacteria of the human digestive and/or urogenital systems, or other bacteria whose byproducts prevent growth of pathogenic bacteria, fungi, and/or yeasts. In one embodiment of this invention the bacteria source 14 contains bacteria beneficial to the vagina or vaginal tract. In the vaginal tract, the bacteria source 14 produces metabolic byproducts that result in a vaginal environment which kills and/or inhibits the growth of pathogenic organisms. Metabolic byproducts such as lactic acid and hydrogen peroxide diffuse from the bacteria source 14 through the outer cover 12 into the vaginal tract limiting pathogenic organism growth and allowing the natural, beneficial organisms normally present in the vaginal tract to flourish.

In one embodiment of this invention bacteria source 14 contains bacteria from the genera Lactobacillus, Bifidobacteria, or combinations of these. In one embodiment of this invention Lactobacillus acidophilus is used in the bacteria source 14. Other genera and various species of bacteria can be used depending on the needs of the treatment and the metabolic byproducts desired.

In one embodiment of this invention inactivated the bacteria source 14 is reactivated by vaginal fluids once the vaginal insert 10 is placed in the vaginal tract. The bacteria source 14 can also be rehydrated outside the vaginal tract by water or various solutions. The bacteria source 14 is desirably activated immediately before insertion into the vaginal tract. A dry powder food source may be mixed with the bacteria source 14 within the outer cover 12 or a food source may be in solution for rehydrating and stimulating metabolic production. Examples of food sources include without limitation fructoogliosaccharides, glycogen, and combinations thereof. In one embodiment of this invention it is desirable that the food source does not also stimulate the infectious pathogenic agent in the vaginal tract.

In one embodiment the food source is intermixed with inactive freeze-dried the bacteria source 14 within the outer cover 12. When the vaginal insert 10 is rehydrated prior to or during use, the bacteria source 14 feeds on the food source and thereby produces the desired metabolic byproducts. The food source can also be in solution and contained separate from the bacteria source 14. In one embodiment of this invention the food source is in solution enclosed within a small plastic blister within the outer cover 12. Before use, the blister is located by touch and squeezed between the user's fingers. The solution containing the food source is released into the bacteria source 14 rehydrating and feeding the inactivated bacteria. In another embodiment a dry food source is contained with inactive the bacteria source 14 and the blister contains only a rehydrating liquid such as, without limitation, water or 0.9 percent by weight sodium chloride.

FIG. 3 shows a blister pack 30 useful for storing and activating the vaginal inserts 10 of this invention. In one embodiment of this invention the blister pack 30 has a first blister 32 containing the vaginal insert 10. A second blister 34 holds a hydrating solution 36. The second blister 34 could additionally hold other desired additives, such as moisture agents. The first blister 32 is connected to the second blister 34 by a blister channel 38. A channel seal 40 keeps the materials in the two blisters from contacting. Before the vaginal insert 10 is used the user would squeeze the second blister 34, causing the hydrating solution 36 to break through the channel seal 40 and enter the first blister 32. The hydrating solution 36 contacts the bacteria source 14 and rehydrates the freeze-dried bacteria therein. The vaginal insert 10 is then inserted into the user's vaginal tract to begin treatment. The hydrating solution 36 can also contain a food source such as fructoogliosaccharides.

Moisture agents can also be contained in the vaginal insert 10 or the hydrating source 36. Moisture agents are additives that impart moisture to dry vaginal tissue. Vaginal dryness often accompanies a vaginal yeast or pathogenic bacterial infection. Examples of moisture agents include without limitation humectants such as propylene glycol, and glycerine, and hydrophilic polymers such as polyvinyl alcohol. Generally compounds used in topical lotions would be beneficial as a moisture agents. Moisture agents can also be enclosed within a blister under the outer cover 12, or otherwise introduced to the vaginal insert 10 just prior to use.

### EXAMPLE

To demonstrate the use of this invention in treatment of vaginal tract infections the following experiment was performed. Freeze-dried Lactobacillus acidophilus (ATCC 4356) was grown in growth media containing 100 grams skim milk, 100 milliliters filtered tomato juice, 5 grams yeast extract, and distilled water to 1 liter, as recommended by ATCC. After 24 hours, 5 microliters of the growth media was diluted to 100 microliters and plated onto media of the same composition plus 1.5 percent by weight agar. After 24 hours, one colony was picked and grown in 6 milliliters growth media for 36 hours. Concentration, as determined by counting by hemacytometer, was 1.37×10⁸ bacteria/milliliters.

A concentrated alginate solution consisting of 3 percent by weight Alginic acid sodium salt, designated as Fluka 71238, available from Fluka BioChemika, Switzerland, in distilled water was prepared. A SPECTRA/POR® dialysis membrane, available from Spectrum Laboratories, California, with a molecular weight cut off of 3,500 and a 11.5 mm diameter was hydrated in sterile water.

At the start of the experiment, 250 microliters of bacteria suspension was placed into three tubes, each containing 2 milliliters growth media, to serve as positive controls. Additional tubes containing only growth media served as negative controls. 750 microliters of suspension was mixed with 750 microliters alginate solution, to create a 1.5 percent by weight alginate solution. This was added dropwise to a mechanically agitated 1.5 percent by weight solution of calcium chloride, designated as Aldrich 23,922-4 from Sigma-Aldrich, Inc., in sterile distilled water. The resulting microparticles measured approx 1.5 millimeters in diameter, and were collected on a sterile filter. These were divided among 3 tubes containing 2 milliliters of growth media each, so the initial number of bacteria was the same as in the positive controls. Blank microparticles were prepared in the same manner, substituting sterile growth media for the bacteria suspension. These were added to 3 tubes, each containing 2 milliliters growth media as negative controls.

A volume of 750 microliters of each of the samples was placed inside the sealed dialysis membrane, which was immersed in 30 milliliters growth media in a sterile media bottle. The samples and controls were placed in a 37° C. shaking incubator. After 8 hours, samples were taken from each tube and the media outside of the dialysis membrane, filtered through a 0.22 micrometer syringe filter, and frozen at −70° C.

The microparticle formation process was repeated with another 750 microliters of bacteria suspension and 750 microliters 3 percent by weight alginate solution. The resulting microparticles were flash frozen in liquid nitrogen. The microparticles were removed from the liquid nitrogen, and placed in a lyophilization flask packed in dry ice. The jar was connected to the lyophilizer, and the microparticles were freeze-dried over night. The next day, the dried microparticles were added to 3 tubes, each containing 2 milliliters growth media. The tubes were placed in the 37° C. shaking incubator for 8 hours. Samples were removed, filtered, and frozen at −70° C.

The following day, the samples were thawed and analyzed for peroxide content using a method published by Fontaine, E. A., and D. Taylor-Robinson, Comparison of Quantitative and Qualitative Methods of Detecting Hydrogen Peroxide Produced by Human Vaginal Strains of Lactobacilli, Journal of Applied Bacteriology 1990, (69) pp. 326-331, herein incorporated by reference. The peroxide concentrations were calculated from a calibration curve and values for appropriate blanks were subtracted. The results were normalized by initial bacteria number and media volume and are expressed as “milligrams H₂O₂ per 1×10⁸ bacteria”. The results show that the positive control produced 0.0148+/−0.0024 milligrams H₂O₂, the bacteria encapsulated in microparticles produced 0.0133+/−0.0028 milligrams H₂O₂, the bacteria which were encapsulated and freeze-dried produced 0.0044+/−0.0022 milligrams H₂O₂, and the bacteria in the dialysis membrane produced 0.0127 milligrams H₂O₂. The results indicate that encapsulation and freeze-drying of Lactobacilli is a promising technique for hydrogen peroxide production which may be used in vaginal applications.

While the embodiments of the invention described herein are presently preferred, various modifications and improvements can be made without departing from the spirit and scope of the invention. The scope of the invention is indicated by the appended claims, and all changes that fall within the meaning and range of equivalents are intended to be embraced therein.

We claim:

1. An apparatus for treatment of a vaginal tract, comprising:
a bacteria source; and
an outer cover surrounding the bacteria source.

2. The apparatus of claim 1, wherein the outer cover is impermeable as to the bacteria source and permeable as to metabolic byproducts of the bacteria source.

3. The apparatus of claim 2, wherein the bacteria source comprises inactive bacteria.

4. The apparatus of claim 3, wherein the inactive bacteria comprises freeze-dried bacteria.

5. The apparatus of claim 3, wherein the bacteria source comprises a powder.

6. The apparatus of claim 3, wherein the bacteria source comprises encapsulated bacteria.

7. The apparatus of claim 5, wherein the outer cover comprises a microporous membrane.

8. The apparatus of claim 7, wherein the outer cover comprises at least one of cellulose, thermoplastic nonwoven webs, thermoplastic films, dialysis membranes, and combinations thereof.

9. The apparatus of claim 6, wherein the outer cover comprises a cloth cover.

10. The apparatus of claim 9, wherein the cloth cover comprises fibers selected from the group consisting of natural fibers, nonwoven fibers, and combinations thereof.

11. The apparatus of claim 1, further comprising a food source within the outer cover.

12. The apparatus of claim 1, further comprising a moisture agent within the outer cover.

13. The apparatus of claim 11, wherein the food sources comprises a compound selected from the group consisting of fructoogliosaccharides, glycogen, and combinations thereof.

14. The apparatus of claim 13, wherein the food source is enclosed and not contacting the bacteria source.

15. The apparatus of claim 12, wherein the moisture agent comprises a compound selected from the group consisting of propylene glycol, glycerine, polyvinyl alcohol, and combinations thereof.

16. The apparatus of claim 15, wherein the moisture agent is enclosed and not contacting the bacteria source.

17. The apparatus of claim 1, wherein the bacteria source comprises bacteria selected from the genera Lactobacillus, Bifidobacteria, and combinations thereof.

18. The apparatus of claim 17, wherein the bacteria source comprises Lactobacillus acidophilus.

19. The apparatus of claim 1, further comprising a string attached to the outer cover.

20. An apparatus for treatment of a vaginal tract, comprising:
a bacteria source encapsulated in alginate microspheres; and
an outer cover surrounding the encapsulated bacteria source wherein the outer cover is impermeable as to the encapsulated bacteria source and permeable as to metabolic byproducts of the bacteria source.

21. The apparatus of claim 20, wherein the bacteria source comprises inactive freeze-dried bacteria.

22. The apparatus of claim 20, wherein the outer cover comprises a cloth cover.

23. The apparatus of claim 22, wherein the cloth cover comprises fibers selected from the group consisting of natural fibers, nonwoven fibers, and combinations thereof.

24. The apparatus of claim 20, further comprising a food source within the outer cover.

25. The apparatus of claim 24, wherein the food sources comprises a compound selected from the group consisting of fructoogliosaccharides, glycogen, and combinations thereof.

26. The apparatus of claim 24, wherein the food source is enclosed and not contacting the bacteria source.

27. The apparatus of claim 20, further comprising a moisture agent within the outer cover.

28. The apparatus of claim 27, wherein the moisture agent comprises a compound is selected from the group consisting of propylene glycol, glycerine, polyvinyl alcohol, and combinations thereof.

29. The apparatus of claim 20, wherein the bacteria source comprises bacteria selected from the genera Lactobacillus, Bifidobacteria, and combinations thereof.

30. The apparatus of claim 29, wherein the bacteria source comprises Lactobacillus acidophilus.

31. A method for treatment of the vaginal tract, comprising:
inactivating a bacteria source;
placing the bacteria source within an outer cover of a vaginal insert, wherein the outer cover is impermeable to the bacteria source and permeable to beneficial metabolic byproducts of the bacteria source;
activating the bacteria source; and
inserting the vaginal insert into a vaginal tract.

32. The method of claim 31, wherein the bacteria source is inactivated by one of lyophilization and sporulation.

33. The method of claim 31, further comprising encapsulating the bacteria source before inactivating.

34. The method of claim 31, wherein the outer cover comprises a microporous membrane.

35. The method of claim 34, wherein the outer cover comprises at least one of cellulose, thermoplastic nonwoven webs, thermoplastic films, dialysis membranes, and combinations thereof.

36. The method of claim 33, wherein the outer cover comprises a cloth cover.

37. The method of claim 36, wherein the cloth cover comprises fibers selected from the group consisting of natural fibers, nonwoven fibers, and combinations thereof.

38. The method of claim 31, further comprising placing a food source with the bacteria source.

39. The method of claim 38, wherein the food source comprises a compound is selected from the group consisting of fructoogliosaccharides, glycerin, and combinations thereof.

40. The method of claim 31, further comprising placing a moisture agent with the bacteria source.

41. The method of claim 40, wherein the moisture agent comprises a compound is selected from the group consisting of propylene glycol, glycerine, polyvinyl alcohol, and combinations thereof.

42. The method of claim 41, wherein the moisture agent is enclosed and not contacting the bacteria source.

43. The method of claim 31, further comprising introducing a moisture agent prior to inserting the vaginal insert.

44. The method of claim 31, wherein the bacteria source comprises bacteria selected from the genera Lactobacilli, Bifidobacteria, and combinations thereof.

45. The method of claim 44, wherein the bacteria source comprises Lactobacillus acidophilus.

46. The method of claim 3 1, wherein the vaginal insert is inserted by a tampon applicator.

47. The method of claim 31, further comprising removing the vaginal insert.

48. The method of claim 47, wherein the vaginal insert comprises a string attached to the outer cover.

49. The method of claim 31, further comprising activating the bacteria source just prior to insertion.

50. The method of claim 31, further comprising packaging the vaginal insert and an activating liquid in a blister pack comprising a first blister for the vaginal insert and a second blister for the activating liquid, wherein the first and second blisters are connected by a sealed blister channel prior to activation.

51. The method of claim 50, wherein activation of the bacteria source is accomplished by squeezing the second blister and breaking the blister channel seal.
